(19) United States
(12) Patent Application Publication
(10) Pub. No.: US 2026/0273118 A1
(43) Pub. Date: Sep. 17, 2026

(54) FLYSWATTER HANGER

(71) Applicant: Jesse Mungia, JR., Gonzales, LA (US)

(72) Inventor: Jesse Mungia, JR., Gonzales, LA (US)

(21) Appl. No.: 19/557,757

(22) Filed: Mar. 5, 2026

Related U.S. Application Data
(60) Provisional application No. 63/834,336, filed on Mar. 17, 2025.

(51) Int. Cl. A61L 2/18

(52) U.S. Cl. CPC A61L 2/18; A61L 2202/121; A61L 2202/122; A61L 2202/15

(57) ABSTRACT

Flyswatter hangers are disclosed that include a container with an internal hanger and an external hanger. Examples include versions with an interior container within an exterior container arranged such that the interior container holds the body of a flyswatter while the flyswatter hangs from the internal hanger. Differing types of applicators are described for applying disinfectant to the flyswatter body along with various configurations of the interior container relating to sanitizing the flyswatter body.

This application claims the benefit of provisional application No. 63/834,336 filed on Mar. 17, 2025 and entitled Flyswatter Holder.

Flyswatter hangers disclosed herein may be used in the storage, sanitary handling, cleaning and sanitization of flyswatters. Certain flyswatter hangers disclosed herein provide convenient storage that repeatedly applies sanitizer to a flyswatter being stored.

### BRIEF DESCRIPTION OF DRAWINGS

FIG. 1 is a drawing of a flyswatter hanger holding a flyswatter.

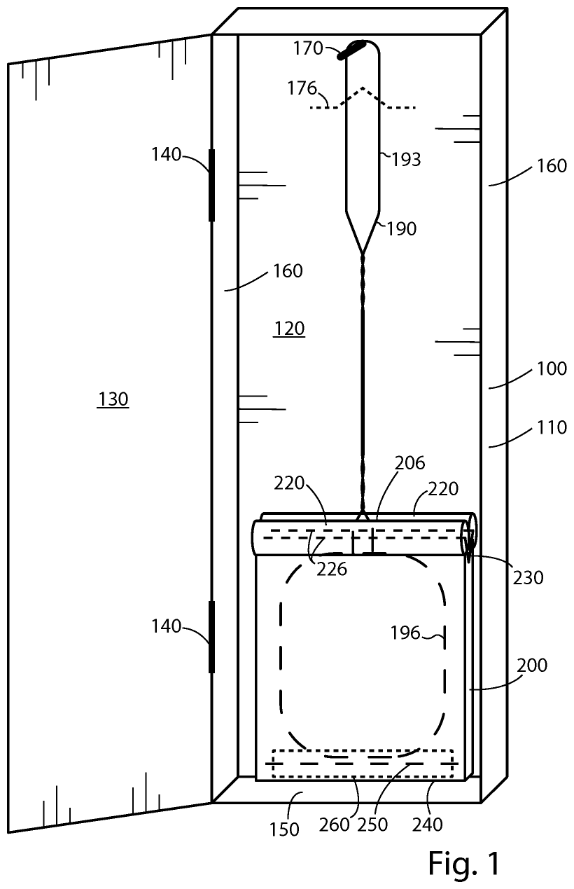

FIG. 2 is a drawing of a flyswatter hanger holding a flyswatter.

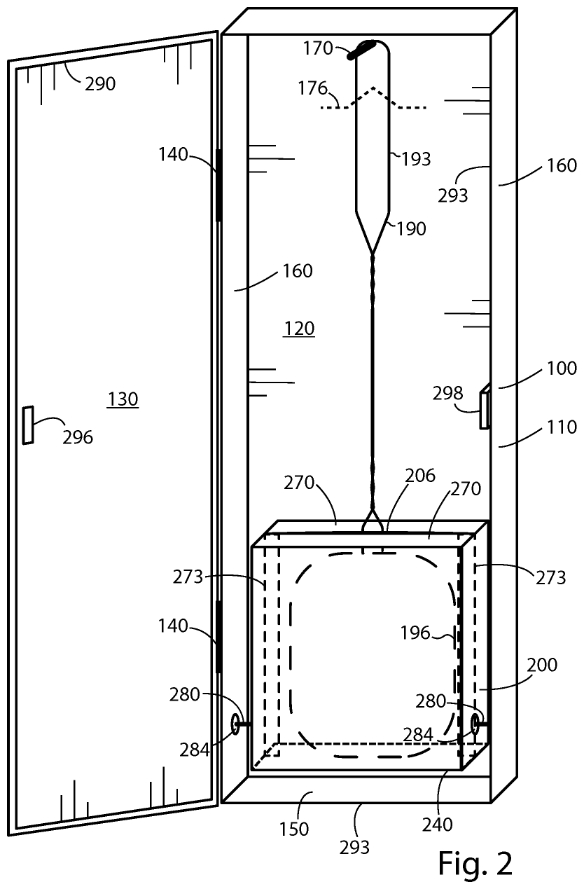

### DETAILED DESCRIPTION

The flyswatter hanger may be configured in accordance with embodiments depicted in the drawings with the following elements associated with the flyswatter hanger:
flyswatter hanger 100,
exterior container 110,
rear wall 120,
door 130,
hinges 140,
exterior container base 150,
exterior container walls 160,
internal hanger 170,
external hanger 176,
flyswatter 190,
flyswatter handle 193,
flyswatter body 196,
interior container 200,
swatter insertion slot 206,
rollers 220,
roller pins 226,
roller spring 230,
interior container bottom 240,
support slot 250,
support tab 260,
transfer pads 270,
interior container internal dividers 273,
pivot pins 280,
pivot pin supports 284,
door lip 290,
door frame sealing edge 293,
door magnet 296, and
frame magnet 298.

Flyswatter hangers described herein may be used to hang a flyswatter. The flyswatter typically has a flyswatter handle and a flyswatter body. The exterior container with an associated door may be configured to completely encompass a flyswatter. The exterior container may have exterior container walls including a rear wall and an exterior container base and may be connected to a door by hinges. An internal hanger may be configured to hang a flyswatter within the exterior container and an external hanger may be configured to hang the exterior container on a wall. An interior container may be configured within the exterior container and generally positioned such that a flyswatter hanging from the internal hanger would have a substantial portion of the flyswatter body covered by the interior container. Insertion of the flyswatter body into the interior container may be done as a first step in storing the flyswatter followed by hanging the flyswatter handle on the internal hanger and closing the door. The flyswatter body may be inserted into the interior container through a swatter insertion slot. The swatter insertion slot may include rollers rotating on roller pins that may be held in position by a roller spring. The roller spring may be configured to apply pressure to the rollers such that disinfectant contained in the rollers is applied to the flyswatter body when the flyswatter body passes between the rollers. In certain embodiments the top of the interior container may tip away from the rear wall such that it presents the swatter insertion slot to the user at a more ergonomic angle for inserting the flyswatter. In one such embodiment, a support slot is present on the rear wall and a support tab attached to the interior container is arranged in that support slot. In that embodiment, the interior container may fall slightly forward and slightly down until the interior container bottom contacts the exterior container base which acts as a stop. In another embodiment, two transfer pads, which may be synthetic sponges are arranged face-to-face inside the interior container, thereby creating the swatter insertion slot. The transfer pads may be replaceable and interior container internal dividers may be used to aid in placement of the transfer pads, to maintain the configuration of the swatter insertion slot, or both. The transfer pads may be similarly used to apply disinfectant to the flyswatter body. The tilting of the interior container may also be by allowing the interior container to pivot relative to the exterior container on pivot pins. Pivot pin supports may be used to stabilize and support the rotation associated with the interior container. The door may create a seal against the other portions of the exterior container preserving the lifespan of the disinfectant. A variety of sealing mechanisms may be used for this purpose including magnets, latches, snapping seals, such as those found in food storage containers, and other similar methods. For example, a door lip may be configured to slide past a snugly fitting door frame sealing edge when the door closes causing a tighter fit than would otherwise be present. Similarly, a door magnet may be used in conjunction with a frame magnet to hold the door tightly against the exterior container when the door is closed.

The exterior container along with the door may take the form of a six-sided box that is capable of completely enclosing a flyswatter within. The height of the exterior container may for example be 17 inches. In many cases the height of the exterior container is at least 12 inches. The height of the exterior container may be less than 24 inches. The width of the exterior container may be between 4.0 and 8.0 inches. The depth of the exterior container may be between 1.0 and 3.0 inches.

The exterior container may be made from a variety of materials including wood and plastic. The interior container may be made from a material such as plastic that is resistant to damage from solvents used in association with disinfectants selected for use on the applicator.

The interior container may be removable and it may be disposable. The interior container may be replaceable such that a used interior container may be discarded into the trash and replaced with a new interior container. The interior container may be between 4.0 and 8 inches tall. The interior container may be between 3.5 and 7 inches wide. The depth of the interior container may be between 0.3 inches and 2.5 inches.

The interior container may be tiltable forward or backward or both. Tilting of the interior container backward indicates that the top of the interior container is tilted out of a vertical orientation toward the rear wall of the exterior container. In cases where the interior container is configured to tilt backward, the angle of backward tilt from vertical may be between 2° and 20°. Tilting of the interior container forward indicates that the top of the interior container is tilted out of a vertical orientation away from the rear wall of the exterior container generally toward a user. In cases where the interior container is configured to tilt forward, the angle of forward tilt from vertical may be between 2° and 60°. The angular range of motion associated with tilting of the interior container from the forward most tilt to the backward most tilt may be between 10° and 50°. The interior container may be stable in an upright position such that the door may close without touching the interior container or the interior container may be configured such that the closing of the door causes contact with the interior container thus returning the interior container to a position fully within the exterior container.

Portions of the flyswatter hanger that may hold disinfectant in a manner that applies the disinfectant to the flyswatter body may be referred to as the applicator or referred to as applicators. The applicator may include a compressible material such as a synthetic sponge material. The applicator may include an absorbent material such as cloth or a sponge material. The user may reapply disinfectant to the applicator by spraying the applicator with an aerosol disinfecting spray.

The applicator may take the form of rollers or may take the form of transfer pads. The applicator may alternatively include other absorbent materials of different shapes and sizes. The applicator may take the form of a full-length applicator, which is capable of making contact with the full-length of a stored flyswatter or the applicator may take the form of a pass-through applicator, which is capable of making contact with the length of a stored flyswatter body as the flyswatter body passes through the applicator in the process of inserting the applicator into the interior container.

Applicators may be adjacent to one another such that the body of the flyswatter is sandwiched between the applicators. The applicators may be zero clearance in that the applicators touch one another when there is no flyswatter in between the applicators. In the case of zero clearance applicators the applicators would separate enough to accommodate the flyswatter body during the insertion of the flyswatter body. Small aperture adjacent applicators may be used such that the clearance between the applicators is less than the thickness of a flyswatter body. In the case of small aperture adjacent applicators, the flyswatter body would also be inserted between the applicators further separating those applicators. The clearance between the applicators without a flyswatter inserted may for example be less than 0.05 inches and, in many cases, may be less than 0.02 inches.

Depending on the configuration of the applicators the contact of the applicators with the flyswatter body as the flyswatter body passes in and out of the interior container may be characterized as rolling contact, sliding contact, or brushing contact.

The applicator materials may be configured to hold, absorb and apply disinfecting spray such as Lysol brand disinfectant spray to the flyswatter body. Alcohols, quaternary ammonium compounds, and other disinfecting liquids may be present on the applicator either by user application, as the initial configuration or both. Alkyl Dimethyl Benzyl Ammonium Saccharinate is an example quaternary ammonium compounds and quaternary ammonium compounds that evaporate more slowly than Alkyl Dimethyl Benzyl Ammonium Saccharinate may be used. Accordingly, the applicator materials may be resistant to chemical solvents such as alcohol and quaternary ammonium compounds. The user may apply disinfectant spray to the applicators after each use of the flyswatter or intermittently. Accordingly, the applicator may hold disinfectant for days, months, or indefinitely. Passing the flyswatter body through the swatter insertion slot may wet the surface of the flyswatter body with disinfectant such that bacteria or viruses present on the flyswatter are killed.

There may be clearance between the sidewall of the interior container and the sidewall of the exterior container. That clearance may be greater than 0.1 inches.

In certain embodiments, such as those utilizing the support tab and support slot configuration, the interior container bottom may be above and out of contact with the exterior container base when the flyswatter hanger is out of use with the door closed. However, opening the door may cause the interior container to fall forward and downward a small amount such that the interior container is angled outward and the interior container bottom makes contact with the exterior container base. This tilting may accommodate easy insertion and removal of the flyswatter. The tilting may also be accomplished manually by pulling the flyswatter handle forward as may be the case when pivot pins are utilized. Accordingly, the interior container may be configured to move within the exterior container such that a user pulling the flyswatter handle would be able to easily move the flyswatter handle toward the user without bending the flyswatter handle.

The flyswatter hanger may be a double hanger flyswatter hanger in that the flyswatter hanger contains both an internal hanger on the interior of the exterior container and an external hanger on the exterior of the exterior container such as may the used to hang the flyswatter hanger on a wall while hanging a flyswatter inside. The flyswatter hanger may also be characterized by the nesting nature of the containers with the interior container arranged in configured for storage entirely within the exterior container.

The door may be configured to have a height that completely hides the flyswatter obscuring both the flyswatter handle and the flyswatter body. When the flyswatter body is in the interior container and the door is open, the flyswatter body may be completely obscured. The door may have an outward facing ornamental appearance, such as a picture, a drawing, or a pattern. The ornamental appearance may be configured such that there is no outward indication that the flyswatter hanger is a flyswatter container.

In cases where the flyswatter body brushes against or slides against the applicator, the applicator may serve a cleaning function by scrubbing the flyswatter body upon insertion or removal of the flyswatter body from the interior container.

The swatter insertion slot may be configured to easily accept a flyswatter by presenting an opening large enough for easily initiating the insertion. As in the case of the rollers, the swatter insertion slot may have and opening wide enough to accommodate and accept a flyswatter that is out of alignment with the swatter insertion slot such that flyswatters may be inserted easily without perfect alignment.

Various configurations may be used to create some level of seal between the door frame sealing edge and the door. For example, a latch may be used. Sealing mechanisms need not create a seal that totally prevents vapors from escaping the flyswatter hanger when the door is closed. A sealing mechanism may decrease the rate of evaporation of solvent from within the exterior container as compared to the rate of evaporation that would exist without the sealing mechanism. This comparison is of evaporation rates when the door is closed and any sealing mechanism that decreases the maximum evaporation rate by more than 5% is considered a “disinfectant conserving sealing mechanism.” Disinfectant conserving sealing mechanisms prolong the length of time for which the applicator is effective at disinfecting the flyswatter.

When the door is closed the flyswatter hanger conceals the contents of the box, namely the flyswatter. As a result, the flyswatter hanger may be placed in open areas, such as in a kitchen, as opposed to being hung in a less accessible place such as a closet.

The above-described embodiments have a number of independently useful individual features that have particular utility when used in combination with one another including combinations of features from embodiments described separately. There are, of course, other alternate embodiments which are obvious from the foregoing descriptions, which are intended to be included within the scope of the present application.

I claim:

1. A flyswatter hanger comprising:
a. an exterior container;
b. a first hanger both internal to the exterior container and attached to the exterior container;
c. a second hanger both external to the exterior container and attached to the exterior container;
d. an interior container arranged and configured to hold the body of a flyswatter hung from the first hanger;
e. an applicator attached to the interior container; and
f. an insertion slot arranged and configured to allow passage of a flyswatter body of the flyswatter into and out of the interior container;
g. wherein the exterior container and the door are arranged and configured together to form a substantially complete enclosure;
h. wherein the applicator comprises a first material;
i. wherein the first material is a compressible material; and
j. wherein the first material is an absorbent material.

2. The flyswatter hanger of claim 1, wherein the interior container is attached to the exterior container such that the interior container may tilt forward when the door is open.

3. The flyswatter hanger of claim 1, wherein the applicator comprises two sponges.

4. The flyswatter hanger of claim 1, wherein the applicator comprises two pads.

5. The flyswatter hanger of claim 1, wherein the applicator comprises two rollers.

6. The flyswatter hanger of claim 1, wherein the door is arranged and configured to completely obscure the interior of the exterior container when the door is closed.

7. The flyswatter hanger of claim 1, wherein the exterior container is at least 12 inches tall and between 4.0 and 8.0 inches wide.

8. A flyswatter hanger comprising:
a. a container;
b. a first hanger both internal to the container and attached to the exterior container;
c. a second hanger both external to the container and attached to the container;
d. a door;
e. an applicator within the container; and
f. a disinfectant located on the applicator
g. wherein the container and the door are arranged and configured together to form a substantially complete enclosure; and
h. wherein the applicator is arranged and configured to transfer a portion of the disinfectant to a flyswatter when the flyswatter is stored in the container.

9. The flyswatter hanger of claim 8, wherein the applicator comprises two sponges.

10. The flyswatter hanger of claim 8, wherein the applicator comprises two pads.

11. The flyswatter hanger of claim 8, wherein the applicator comprises two rollers.

12. The flyswatter hanger of claim 8, wherein the door is arranged and configured to completely obscure the interior of the container when the door is closed.

13. The flyswatter hanger of claim 8, wherein the container is at least 12 inches tall and between 4.0 and 8.0 inches wide.

14. A flyswatter hanger comprising:
a. an exterior container;
b. a first hanger both internal to the exterior container and attached to the exterior container;
c. a second hanger both external to the exterior container and attached to the exterior container;
d. a door;
e. an applicator arranged and configured to apply a disinfectant to a flyswatter;
f. an interior container arranged and configured within the exterior container such that a body of the flyswatter may reside within the interior container while the flyswatter is hanging from the first hanger; and
g. a disinfectant conserving sealing mechanism arranged and configured to enhance sealing between the exterior container and the door;
h. wherein the exterior container and the door are arranged and configured together to form a substantially complete enclosure.

15. The flyswatter hanger of claim 14, wherein the interior container is attached to the exterior container such that the interior container may tilt forward when the door is open.

16. The flyswatter hanger of claim 14, wherein the applicator comprises two sponges.

17. The flyswatter hanger of claim 14, wherein the applicator comprises two pads.

18. The flyswatter hanger of claim 14, wherein the applicator comprises two rollers.

19. The flyswatter hanger of claim 14, wherein the door is arranged and configured to completely obscure the interior of the exterior container when the door is closed.

20. The flyswatter hanger of claim 14, wherein the exterior container is at least 12 inches tall and between 4.0 and 8.0 inches wide.
